(19) United States
(12) Patent Application Publication
(10) Pub. No.: US 2023/0026703 A1
(43) Pub. Date: Jan. 26, 2023

(54) SYSTEM AND METHOD FOR IDENTIFYING TREATABLE AND REMEDIABLE FACTORS OF DEMENTIA AND AGING COGNITIVE CHANGES

(71) Applicant: Eliahu Yosef WERTMAN, Ma'ale Michmas (IL)

(72) Inventor: Eliahu Yosef WERTMAN, Ma'ale Michmas (IL)

(21) Appl. No.: 17/882,070

(22) Filed: Aug. 5, 2022

Related U.S. Application Data
(63) Continuation-in-part of application No. PCT/IL2021/050139, filed on Feb. 5, 2021.

(30) Foreign Application Priority Data
Feb. 5, 2020 (IL) 272496

(51) Int. Cl. G16H 50/20; A61B 5/00; G16H 15/00; G16H 20/00; G16H 50/50; G16H 50/70

(52) U.S. Cl. CPC G16H 50/20; A61B 5/4088; A61B 5/4842; A61B 5/7282; G16H 15/00; G16H 20/00; G16H 50/50; G16H 50/70

(57) ABSTRACT

The present invention relates to a method and system for identifying treatable and remediable factors of Dementia and aging cognitive changes, to provide recommendations for aiding in the diagnosis of dementia or predementia symptoms in a subject. According to an embodiment of the invention, the method comprising: receiving data relative to medical history and examinations, processing said received data by applying an algorithm(s) relative to the Intensive Neuropsychogeriatric Evaluation, Treatment and Prevention (INETAP) method, and verifying whether said processed data is sufficient for indicating of advanced Dementia Potential Remediable Conditions (PRCs), and outputting data for aiding in the diagnosis of one of the following: dementia PRCs, pre-dementia PRCs, no dementia/pre-dementia, or Dementia without treatment horizon.

### CROSS-REFERENCE TO RELATED APPLICATIONS

This application is a Continuation-In-Part Application of International Application Number PCT/IL2021/050139, filed Feb. 5, 2021; which claims priority to Israel Patent Application No. 272496, filed Feb. 5, 2020; both of which are incorporated herein in their entirety.

### FIELD OF THE INVENTION

The present invention relates to the field of medical systems. More particularly, the invention relates to a system and method for identifying treatable and remediable factors of Dementia and aging cognitive changes.

### BACKGROUND OF THE INVENTION

Dementia is a complex phenomenological syndrome with acquired multiple cognitive and behavioral deficits that interfere with everyday activities-occupational, social, and other comportmental functions to the level of loss of independent life and full dependence on caregivers. The last are suffering from chronic stress and a lower threshold to medical conditions.

Symptomatic dementia and predementia states are highly prevalent in the elderly:

Dementia: 5-10% above 65 years old, and 42-76% above 80;

Mild Cognitive Impairment and Subjective Cognitive Impairment: above the age of 65-10-32% and 17%, respectively, depending on methodology and clinical setting.

Currently, it is estimated that there are 50 million people worldwide suffering from Dementia, and the projection for 2030 is 82 million and by 2050, is 152 million (2019 Survey of U.S. Alzheimer's Association) see FIG. 14.

The total estimated worldwide cost of Dementia is $1 trillion (in 2018) and is projected to reach $2 trillion by 2030 (see FIG. 14).

The Following Summarizes Four Different Approaches that Failed:

The failure of current therapeutic approaches to Dementia Syndrome (DS)—up to now, there are no existing treatments for DS. This is true for both specific etio-pathophysiological treatment and for prevention approaches.

### The Lack of Specific Etio-Pathophysiological Treatment in Clinical Symptomatic DS—

A classical therapeutic approach to a clinical medical illness is by either treating its specific etiological causes (e.g., cobalamin deficiency, hypertension-overtreatment hypoperfusion, and as well as other established treatable conditions) to reverse the condition or by a Disease-Modifying Treatment (DMT) of conditions like Alzheimer's Disease (AD)?, Vascular Dementia (VD) and other dementing disorders. However, the reported prevalence of actual reversible etiological conditions is low, less than 1% of the dementia patients. Also, there is no existing clinically relevant DMT for AD and other dementing disorders with a failure of more than 400 clinical trials of medications. DMT for VD needs knowledge of the cellular and molecular components of the vascular tree and its associated cognitive changes. At the moment, we are very far from it, including hypertension, which is the major cause of VD. The same is for other degenerative conditions like Lewy Body Dementia (LBD), Primary Progressive Aphasia, Frontotemporal dementia, Dementia of Parkinson's disease, and other dementing conditions. Developing DMT for pre-dementia conditions is even more difficult due to the lack of identified treatable primary pathophysiological processes and molecules.

### The Failure of the Prevention Approach

The prevention approach is based on the existence of preclinical neuropathological changes for conditions like AD VD and some modifiable risk factors (e.g., cardiovascular, lifestyle, depression). It is assumed that with mid to late-life prophylaxis—up to 51-54% of dementia cases can be prevented worldwide. Late-life prevention is suggested to contribute about 15% to dementia risk reduction.

Some longitudinal cohort studies from Europe and the USA do suggest a decline in age-specific incidence of Dementia. These changes were related mainly to improved cardiovascular prevention in accordance with the Framingham principles. However, some longitudinal intervention studies found no such certain changes. Ambiguity is the rule even for studies like the Finnish Geriatric multi-domain intervention (FINGER) Study. Also, the rates of reported prevention effects are low, compared to the epidemiological threat.

Difficulties in establishing prevention protocol for Dementia show almost no effectiveness due to mainly following reasons:
1) methodological factors such as: a) selection bias, refusal and mortality rates, criteria of dementia b) variability of risk factors and pharmacological treatments, c) neglecting general medical-geriatric changes, and d) lack of generalization power.
2) inability to control tacit factors like: a) the cause of lower effect of vascular prevention on chronic conditions such as CHF and Dementia than on acute cardiovascular disease and b)non-vascular factors like birth year and other macro and meso levels effect.
3) The statistical rather than individual patient-centered directed protocols. The effect of prevention studies like the FINGER prototype is “most appropriately interpreted in a public health context, in which small long-term effects can be highly relevant”. The minimal individual effect is declared by the team of the Finger study themselves.

As a result, the current primary and secondary prevention approach is based on an expert opinion based on unproven impressions rather than on fully evidence-based medicine. As is summed in a 2018 Alzheimer's Association Facts and Figures report— “ . . . treatments to prevent, slow or stop these changes are not yet available, although many are being tested in clinical trials”. This is, of course, in addition to the essential residual high rate of DS—at least 50-60%—in the face of even future successful prevention.

### The Failure to Include Already Symptomatic DS Patients in Curative Treatments Efforts

After 50 years of intensive research since Tomlinson, Roth and Blessed, there is no effective treatment for Dementia, with disastrous negligence of the symptomatic patients. Such treatments are not realistically expected, at least in the coming 10-20 years. Since about 90% of DS happens in elderly people, a stigmatic “age”-istic attitude is taken, including the acceptance of DS as part of normal aging. Meanwhile, patients with symptomatic DS and their families have daily suffered from mental, behavioral, functional, social, and economic decline. This is in addition to the progressive living loss and bereavement of marriage-partners and parents of families. Treatments are mostly directed to intercurrent conditions like infection or agitation, not to a causative process. Thus, there is an urgent need to search for other ways to concretely treat and help the continuously growing numbers of suffering symptomatic dementia patients and their caregivers.

Additional reasons to re-focus on medical treatment of the symptomatic phase of Dementia include—
1) Symptom definition in the symptomatic phase of Dementia is the basis of any clinical search for the treatment of DS.
2) The recent availability of advanced behavioral neurology, neuropsychology, and neuropsychiatry knowledge, as well as the better clinical and technical capabilities to diagnose and find treatments for symptomatic dementia patients and a better symptom-directed etiological treatment.
3) Symptom definition is the basis of clinical research in medicine in general. Also, due to the phenomenological heterogeneity of Dementia, there is a need for the sharp-borders definition of the core and associated symptoms of presenting dementia patients as a basis for both clinical and research approaches.
Thus, efforts to diagnose symptomatic DS patients will be a better basis to try a clinical treatment approach to alleviate their current clinical frustration.

### The Lack of Solid Medical Methods for Clinical Diagnosis of DS

As was mentioned above, the lack of any valid curative and preventive approaches results in the stigmatization of the condition. Consequently, there is frustration among the medical and social communities. They see the condition as a disease that causes a loss of autonomy, without any specific medical treatment, with the main goal to keep the patient at home and deep skepticism about any way to develop help. This has a lot of outcomes, including neglecting classical medical attitude with a proven avoidance of accepted medical diagnostic work-up.

Thus, it is of utmost importance to have a valid method that will be effectively used in daily clinical work. No method like this exists.

It is an object of the present invention to provide a system for identifying treatable and remediable factors of Dementia and aging cognitive changes.

It is another object of the present invention to provide a data analysis system that is capable of automatically creating the foundations of creating more sophisticated thresholds (based on validated clinical data) for further decisions and actions, repeatability of decisions of preferred Potentially Remediable Conditions (PRCs), and providing the statistic foundations of preferred PRCs decisions, thereby making it easier for clinicians to rely on preferred PRCs, allowing a faster authorization of issued preferred PRCs, shorten the training duration of the medical team, and to consider and integrate new published worldwide relevant research and to allow external information feed such as wearables and Internet of Things (IoT) devices.

Other objects and advantages of the invention will become apparent as the description proceeds.

### SUMMARY OF THE INVENTION

The present invention relates to a method for identifying treatable and remediable factors of Dementia and aging cognitive changes, thereby enabling to provide recommendations (e.g., outputting information for clinicians) for aiding in the diagnosis of dementia or predementia symptoms in a subject. The importance of an appropriate diagnosis may lead to treating, stabilization, improvement, and preventing deterioration in subjects diagnosed or having dementia or predementia symptoms. In addition, it may further lead to preventing the appearance of cognitive changes, Dementia, or pre-dementia symptoms before developing dementia or predementia syndromes.

### According to an Embodiment of the Invention, the Method Comprises:

receiving data relative to medical history (e.g., detailed cognitive, behavioral, functional, neurological, psychiatric, lifestyles, psychosocial, medical, and geriatric) and examination (e.g., behavioral neurology, neuropsychology, psychogeriatric, neurology, psychosocial, medical and geriatric);
Processing said received data by applying an algorithm(s) relative to the Intensive Neuropsychogeriatric Evaluation, Treatment and Prevention (INETAP) method and verifying whether said processed data is sufficient for indicating of advanced Dementia Potential Remediable Conditions (PRCs); and
Outputting data for aiding in the diagnosis of one of the following: dementia PRCs, pre-dementia PRCs, no dementia/pre-dementia, or Dementia without treatment horizon.

According to an embodiment of the invention, the method further comprises outputting recommendations in accordance with symptoms of pre-dementia or Dementia PRCs (e.g., providing recommended treatment instructions).

According to an embodiment of the invention, the method further comprises outputting recommendations in accordance with existing risk factors for no dementia/pre-dementia or Dementia without a treatment horizon.

According to an embodiment of the invention, the data comprises genetics, age, resilience, lifestyle, homeostasis & allostasis processes in accordance with multimorbidity-vascular disorders, multimorbidity-systemic disorders, and multimorbidity-geriatric disorders, etc.

According to an embodiment of the invention, the algorithm comprises: a) processing data received from different levels of pathogenetic causality of Late-Onset Dementia (LOD) Syndrome Complex (e.g., a proximal conditions tier, an intermediate systemic tier, etc.), as well as data relative to distal brain molecular and cellular processes, b) identifying pathological changes in accordance with said processed data; and c) providing symptomatic LOD.

In another aspect, the present invention relates to a system for diagnosing and preventing dementia syndrome, comprising:

a) at least one processor; and

b) a memory comprising computer-readable instructions which, when executed by the at least one processor, causes the processor to execute a dementia syndrome agent, wherein the agent:
i. receives data relative to medical history and examinations of a patient (e.g., detailed cognitive, behavioral, functional, neurological, psychiatric, lifestyles, psychosocial, behavioral neurology, neuropsychology, psychogeriatric, psychosocial, medical, and geriatric);
ii. Processes said received data by applying algorithm(s) relative to INETAP method and verifying whether said processed data is sufficient for indicating advanced Dementia PRC or early-stage Dementia PRC; and
iii. Outputs data for aiding in the diagnosis of one of the following: Dementia PRCs, pre-dementia PRCs, no dementia/pre-dementia, or Dementia without treatment horizon.

According to an embodiment of the invention, the PRC agent enables:
to create the foundations of creating more sophisticated thresholds for further decisions and actions;
to create repeatability of decisions of preferred PRCs; and
to provide the statistical foundations of preferred PRCs decisions,
thereby making it easier for clinicians to rely on preferred PRCs, allowing a faster authorization of issued preferred PRCs, shortening the training duration of the medical team, and considering and integrating new published worldwide relevant research, and allowing an external information feed.

According to an embodiment of the invention, in addition to manual inputting/loading of data and available data files loading, the external information feed is also received from available and future developed wearables and Internet of Things (IoT) based devices.

### BRIEF DESCRIPTION OF THE DRAWINGS

The above and other characteristics and advantages of the invention will be better understood through the following illustrative and non-limitative detailed description of embodiments thereof, with reference to the appended drawings, wherein:

FIG. 1 shows disease Hypertension Hierarchical Multilevel Ontology (HMO);

FIG. 2 shows the Multimorbidity (MUM) etiological (past phenomenological syndromes definition) evaluation in Symptomatic Late-Onset Dementia (SLOD);

FIG. 3 schematically demonstrates an example of a PRCs active process course (e.g., medication, hypotension that needs identification and treatment eradication);

FIG. 4 schematically demonstrates an example of a treated PRCs course with enhancing activities;

FIG. 5A schematically demonstrates an example of simple course profiles;

FIG. 5B schematically demonstrates an example of a peri-onset deterioration course with later stability of improvements;

FIG. 6 schematically demonstrates an example of a heterogeneous complex course (on the background of simple continuous monotonous progression);

FIG. 7 schematically demonstrates an example of a heterogeneous complex course with two contingencies (on the background of simple continuous monotonous progression);

FIG. 8 is a chart that shows a treatment sample of 100 patients with no active specific program, i.e., before using the Intensive Neuropsychogeriatric Evaluation, Treatment and Prevention (INETAP) method of the present invention;

FIG. 9 is a chart that illustrates the main categories of active Potentially Remediable Conditions (PRCs) count;

FIG. 10 shows a table of active specific PRCs list, according to an embodiment of the invention;

FIG. 11 is a flow chart of the INETAP method, according to an embodiment of the present invention;

FIG. 12 schematically illustrates a system for identifying treatable and remediable factors of Dementia and aging cognitive changes, according to an embodiment of the invention;

FIG. 13 schematically illustrates, in a block diagram form, an integration of a diagnostic clinic based on the INETAP method with clinical units, according to an embodiment of the invention; and

FIG. 14 schematically illustrates an example for a data analysis process in accordance with the INTEPAP method of the present invention; the data analysis process may involve the following stages:
Stage 1—data input stage 141—in this stage, a list of parameters is filled in, based on medical history, lab tests, consultations, etc. For example, the parameters may include active high grade dynamic systemic/geriatric condition, acute confessional state/delirium, endangering psychiatric condition, gait disorder, urinary loss, rectal loss, life events, CVA, TIA, cataract, goiter, neurologic signs admissions, MRI brain, cognitive disorders, etc. Each parameter receive a corresponding value (e.g., as schematically indicated by the values columns in the figure, which includes values 1-20);
Stage 2— signification filtering 142—in this stage, the system filters parameters of low significance (among the parameters received in stage 1) to make the signature more significant;
Stage 3— signature creation 143—in this stage, a signature is created (i.e., PRC Signature), which is composed of the values of each remaining parameter (after the filtration);
Stage 4—trajectory comparison 144—in this stage, the system provides several options of differential & ethiodiagnosis—prevalence in the database. In other words, the various differential diagnoses are displayed (e.g., as indicated by numerals 144a, 144b, 144c, 144d);
Stage 5—statistic threshold filtering 145—in this stage, differential diagnosis of low similarity is filtered away. In this stage, the user can modify the level of the desired similarity; and
As a result of the above, the system outputs the final differential diagnosis 146a and 146b, thereby allowing clinicians to take action based on similarity with previous cases; and

FIG. 15 shows an infographic expression of the 2019 Survey of U.S. Alzheimer's Association estimating scope of Dementia and associated costs in 2018 and long-term projection.

### DETAILED DESCRIPTION OF THE INVENTION

The present invention provides a method, system, and code for aiding in the diagnosis of dementia or pre-dementia syndrome in a subject. In some embodiments, the present invention relies on the use of a statistical algorithm (e.g., a learning statistical classifier system) and/or empirical data (e.g., data relative to detailed cognitive, behavioral, functional, Neurological, Psychiatric, Life-Styles, Psychosocial, Medical, and Geriatric). The present invention is also useful for ruling out one or more diseases or disorders that present with dementia-like symptoms and ruling in Dementia using a combination of statistical algorithms and/or empirical data. Accordingly, the present invention provides an accurate diagnostic prediction of Dementia Potential Remediable Conditions (PRC) or pre-dementia PRC and prognostic information useful for guiding treatment decisions.

### The Conceptual Basis of the Invention

### Insights

1) Symptomatic Late-Onset Dementia (SLOD)—is the preferred target for diagnosis and treatment of Dementia. This is due to the 95% prevalence of SLOD from dementia patients, its nosological differentiation from young-onset Dementia (background brain changes, different neuropathological and genetic expressions, clinical heterogeneous presentations, systemic multimorbidity, and acceleration of prevalence and course with age).
2) High prevalence of Multimorbidity (MUM)—MUM is highly prevalent in the elderly and is associated with SLOD. There is an increased prevalence of SLOD with a higher number of systemic MUM conditions. MUM conditions are associated with their resulting effect on the lesion of the brain. Most of the MUM conditions in the elderly have the potential to be treated, thus their treatment has the potential to stop or improve SLOD progression, i.e., Potentially Remediable Conditions (PRCs). Importantly, there is a parallel age-associated acceleration of prevalence of both MUM and SLOD. This suggests a causal MUM effect on the appearance and progression of SLOD.
3) The cognitive, behavioral and functional multi-phenomenology of SLOD—SLOD syndrome usually presents with cognitive (like dyslexia without dysgraphia, category-specific anomia and prosopagnosia, behavioral (like depression, agitation, and visual hallucinations), and functional (like deconditioning and disability) co-existing multi-phenomenology. Many of these phenomena are specific sub-syndromes of SLOD and might be caused by specific pathophysiological and neuropathological processes, other than that of a global syndrome (SLODg) and its common pathologies like Advanced Dementia (AD) or Lewy Body Dementia (LBD). They might reflect different MUM conditions and PRCs.
4) The MUM-multi phenomenology complex feature of SLOD—SLOD, including AD, is a complex disorder, which is caused by complex interactions between its components and is hard to explain by few factors. The interactions are in a disordered way, resulting in a powerful level of organization and memory. It entails dynamic features like pleiotropy, robustness, and rewiring. The Complex System (CxS) network features of SLOD have a clinical effect on its phenomenological—syndrome definition and etiological MUM and PRCs identification. This demands a different diagnostic process from the conventional one. The last is based on a reductive approach with identifying syndromic phenotypes, which are correlated with pathological analysis and laboratory tests with a linear chain from pathology to disease. The CxS features of the disease are more suggestive of the multifactorial basis of disease. SLOD is extremely complex due to its heavily dense multimorbidity space and very highly crowded complex phenotypical space. The resulting clinical features are heterogeneous. In addition to the diagnostic difficulty, the complexity of SLOD is enhanced by its highly dynamic course. The causality is non-linear and, therefore, complex. A highly universal and relevant manifestation of a complex system is Emergent Behavior (EB). An EB phenomenon in a CxS is the appearance of whole system behavior. EB emerges from the multiple non-linear interactions between the system's different many components and levels, which integrate into a functioning whole. The interactions exist in a virtually infinite number of states. Through a process called “self-organization”, the system will “naturally” settle into a reduced number of “stable” configurations” which is EB. EB results in the irreducibility of its causes due to the inability to define or predict any of its causal individual parts. This is a factor that decreases the ability to identify the elemental components of SLOD. In fact, SLOD is an EB phenomenon, thus current criteria essentially direct the diagnostic process to its global macro-features and exclude a full definition of co-existing MUM and PRCs that affect the appearance and progression of SLOD. This, of course, ends in a diagnosis of a degenerative or irreversible vascular disorder.
5) In order to issue preferred PRCs, clinicians need to (1) cope with the vast quantity and variation of the information, including PRCs identified, gathered within INETAP, (2) understand the correlations between the various data points, (3) be able to weigh between the importance of such data and (4) compare with previous cases of high similarity. This requires high qualifications, extensive training, continuous follow up of new published worldwide relevant medical research, and lacks repeatability. In addition, clinicians may be biased in recent cases.

### Summary of Insights

It is clear that the current diagnostic work-up of Dementia is wrong. Its components of the gathering of information and medical reasoning are inadequate for a complex system like SLOD, which is the strategic component of Dementia Syndrome (DS).

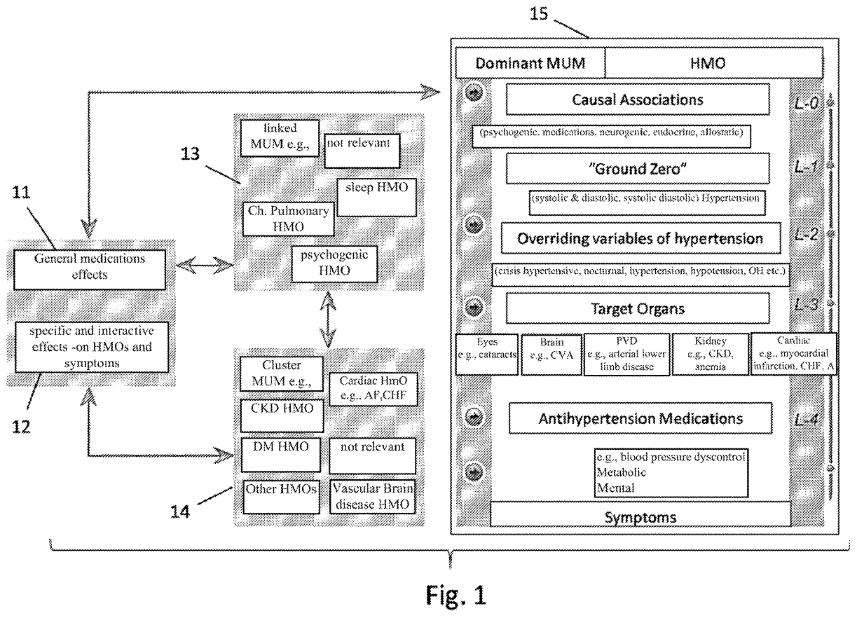

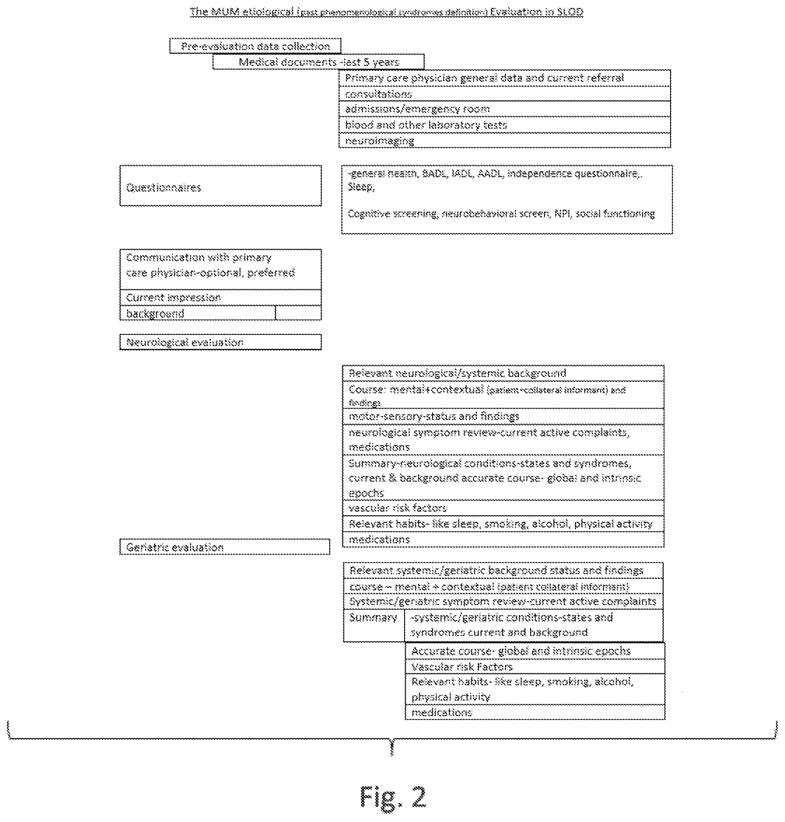

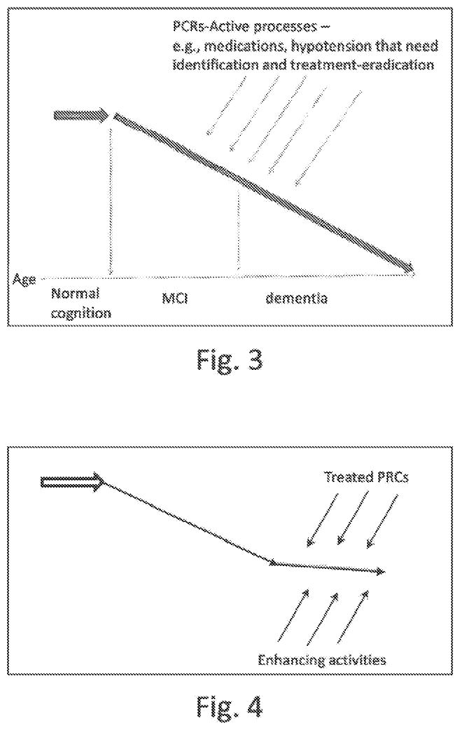

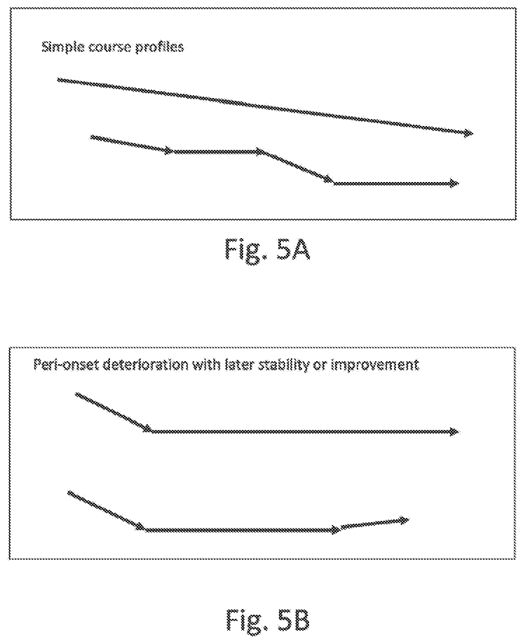

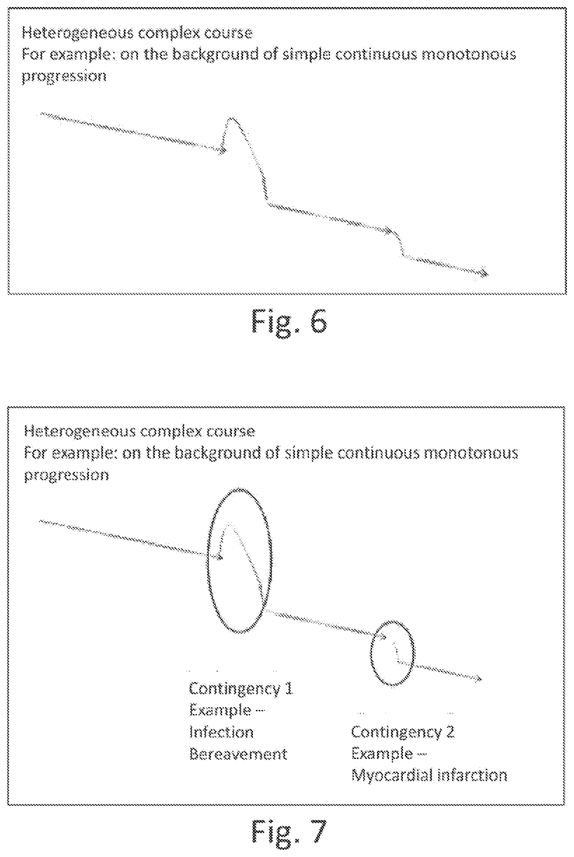

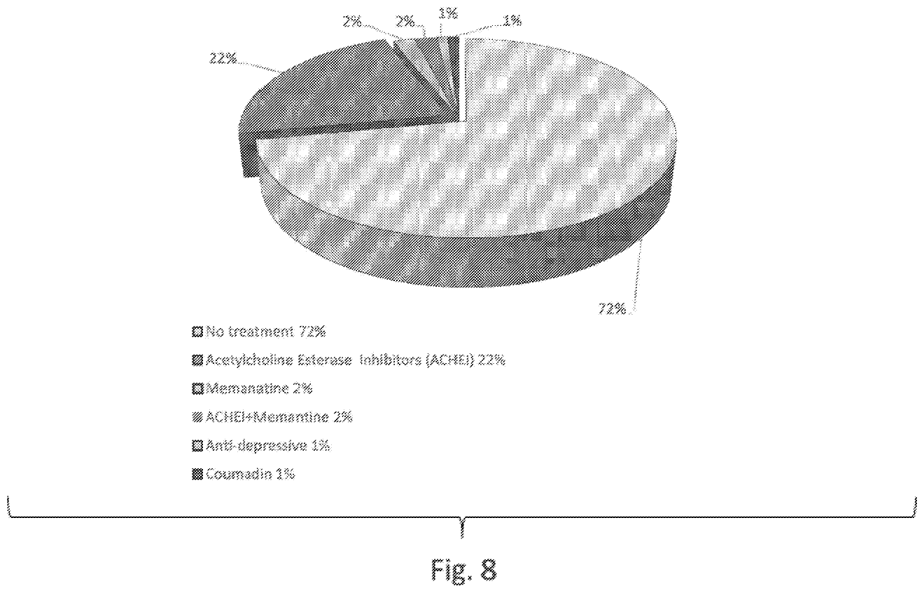

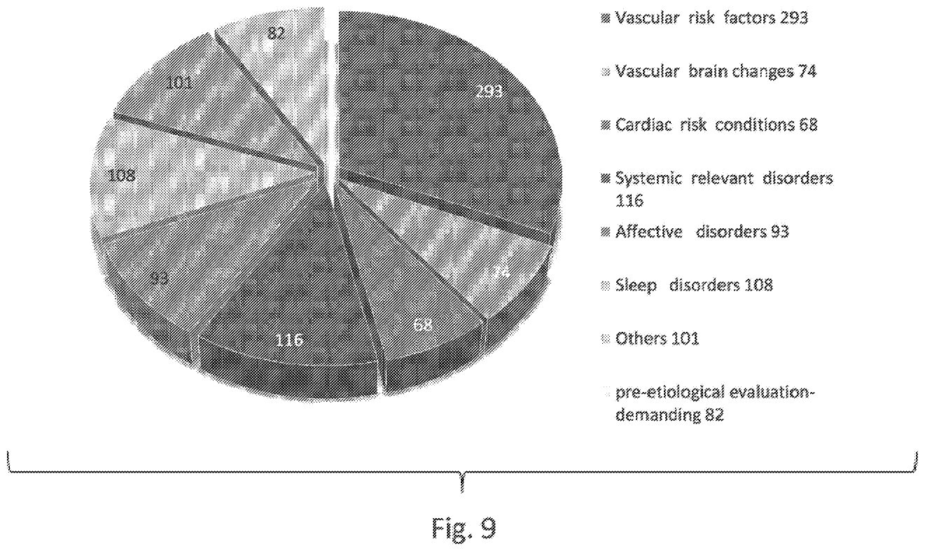

According to an embodiment of the invention, in order to overcome the above-mentioned dimensions of Dementia and to supply a valid solution that will answer its derivative requirements, there is a need to concentrate on SLOD, and to focus on the following elements:
1) fully identify every phenomenological co-existing syndrome (e.g., SLOD itself, co-existing mega-syndromes like Confessional state, depression, etc., and sub-syndromes like optic aphasia, etc.). See FIG. 1 that shows disease Hypertension Hierarchical Multilevel Ontology (HMO) 15, which involves general medications effects 11, specific and interactive effects 12 (on HMOs and symptoms), linked MUM 13 (e.g., sleep HMO, psychogenic HMO, etc.), cluster MUM 14 (e.g., chronic kidney disease (CKD) HMO, cardiac HMO, etc.). For example, the multilevel can may involve several levels (e.g., as indicated by L-0, L-1, L-2, and L-4 in the figure) that form the symptoms. In this example, Level 0 (L-0) refers to causal associations (e.g., psychogenic, medications, neurogenic, endocrine, allostatic), Level 1 (L-1) is the “ground zero”, and it refers to hypertension (systolic and diastolic), Level 2 (L-2) refers to overriding variables of hypertension (crisis hypertensive, nocturnal, etc.), Level 3 (L-3) refers to target organs such as eyes (e.g., cataract), brain (e.g., Cerebrovascular accident—CVA), kidney (e.g., CKD, anemia), cardiac (e.g., myocardial infarction), etc. Level 4 (L-4) refers to antihypertension medications (e.g., blood pressure, metabolic, mental, etc.);
2) a thorough etiological evaluation. For example, see FIG. 2 that shows the MUM etiological (past phenomenological syndromes definition) evaluation in SLOD;
3) based on detailed collecting of information—cognitive behavioral, functional, and medical, both of presentation and previous course of development. (see FIGS. 3-7);
4) an integrated differential diagnosis of every cognitive, behavioral and functional syndrome and sub-syndrome;
5) syndrome-associated PRC determination; and
6) a data analysis system that enables creating the foundations of creating more sophisticated thresholds for further decisions and actions, to create repeatability of decisions of preferred PRCs, to provide the statistic foundations of preferred PRCs decisions, thereby making it easier for clinicians to rely on preferred PRCs, allowing a faster authorization of issued preferred PRCs and shorten training duration of the medical team and to consider and integrate new published worldwide relevant research and to allow external information feed such as wearables and Internet Of Things (IoT).

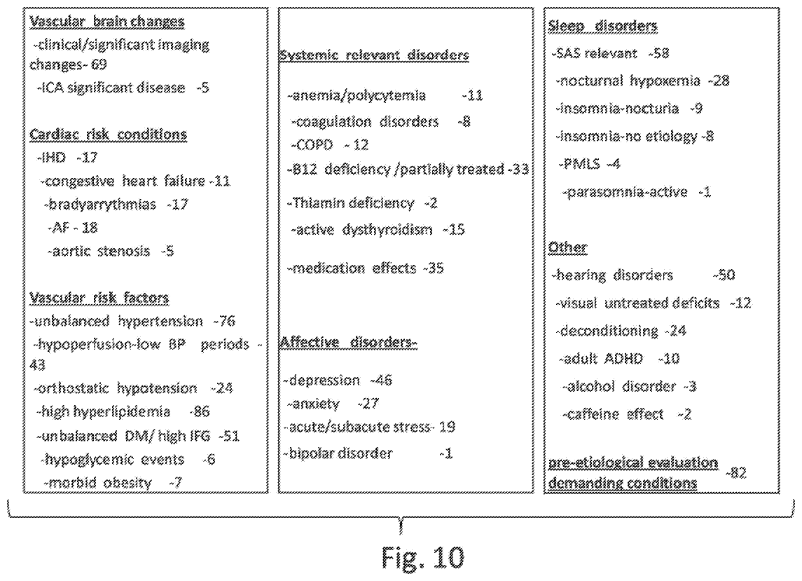

These concepts were developed and integrated by the inventor into an Intensive Neuropsychogeriatric Evaluation, Treatment, and Prevention (INETAP) method (an article by the inventor: “The Multimorbidity, Multiphenomenology and Complexity concept of Symptomatic late-onset Dementia—a Potential for Modifying and Prevention” will be published). Over 4000 patients have been clinically examined so far. Numerous PRCs have been identified in practically all of the patients. Recent sample of 100 patients shows 935 PRCs (9.35 PRCs/patient) (see FIGS. 8-10). Specific treatment was given where needed. Retrospective questioning reveals that about 85% of them either improved or stayed stable for 1-4 years (sometimes even longer). All these patients were evaluated (before using the INETAP method) by several competent memory clinics and received diagnoses of degenerative and irreversible vascular conditions, without any PRCs found or any curing treatment horizon.

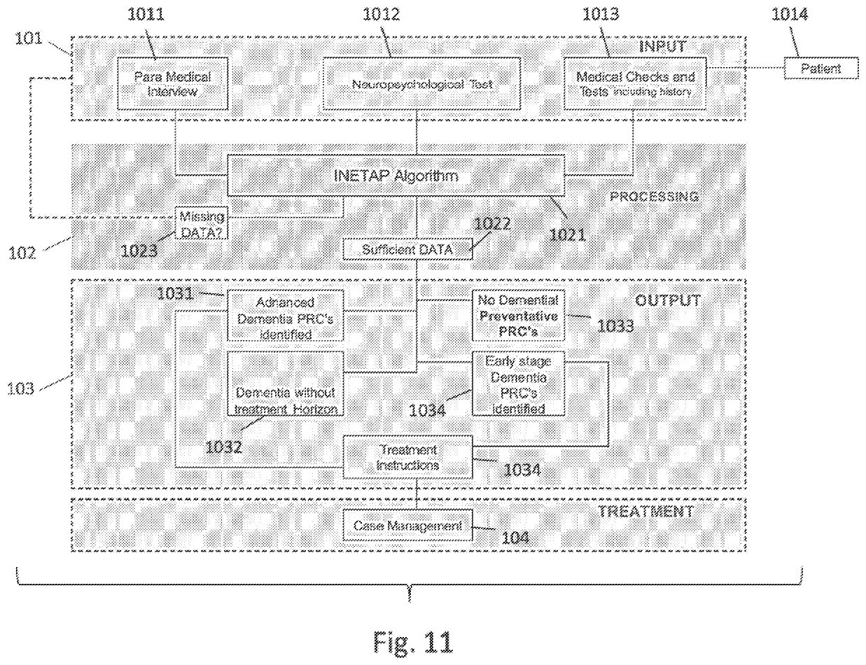

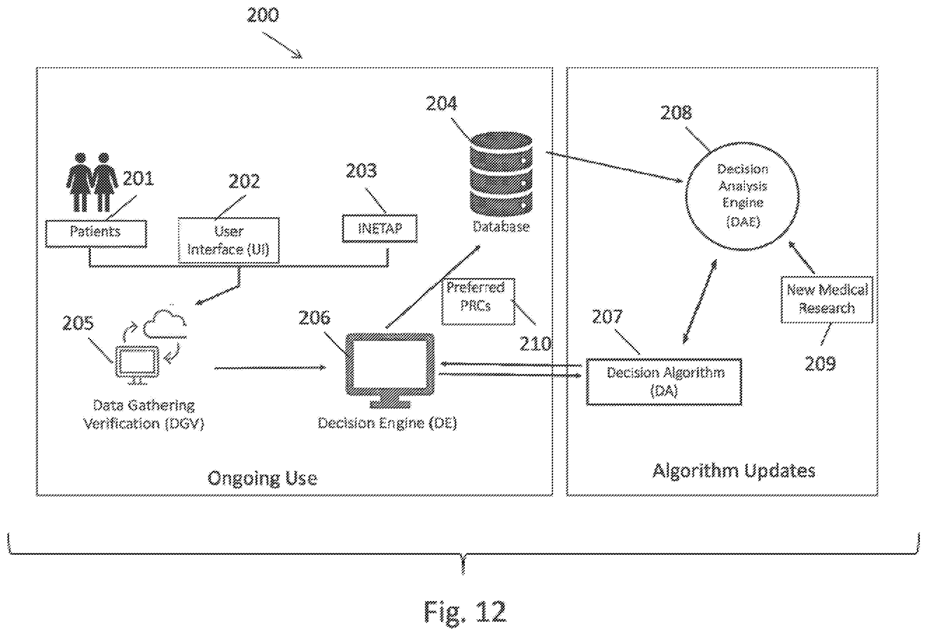

FIG. 8 shows a graph of treatment applied to a sample of 100 patients before using the method of the present invention. As shown in the graph of FIG. 1, 72% of the patients receive no treatment, 22% of the patients treated with Acetylcholine Esterase Inhibitors (ACHEI), 2% of the patients treated with Memantine (i.e., a medication used to treat moderate to severe Alzheimer's disease), 2% of the patients treated with ACHEI and Memantine, 1% of the patients treated with anti-depressive, and 1% of the patients treated with Coumadin (i.e., Warfarin—a medication that is used as an anticoagulant). FIG. 9 shows a graph of the main categories of active PRCs count for the 100 patients (a total of 935 active PRCs and an average of 9-10 active PRCs per LOD/Mild Cognitive Impairment (MCI) patient). As shown in this graph, the active PRCs are distributed as follows: vascular risk factor 293, vascular brain change 74, cardiac risk conditions 68, systemic relevant disorders 116, affective disorders 93, sleep disorders 108, others 101, and pre-etiological evaluation-demanding 82. FIG. 10 shows a table of the active specific PRCs list that includes vascular brain changes (e.g., clinical/significant imaging changes—69 RPCs, ICA significant disease—5 RPCs), cardiac risk condition (e.g., IHD—17 RPCs, congestive heart failure—11 RPCs, bradyarrhythmias—17 RPCs, etc.), vascular risk factors (e.g., unbalanced hypertension—76 RPCs, hypoperfusion-low BP periods—43 RPCs, etc.), systemic relevant disorders (e.g., anemia/polycythemia—11 RPCs, coagulation disorders—8 RPCs, etc.), affective disorders (e.g., depression—46 RPCs, anxiety—27 RPCs, etc.), sleep disorders (e.g., nocturnal hypoxemia—28 RPCs, insomnia-nocturia—9 RPCS, etc.), pre-etiological evaluation demanding conditions (e.g., 82 RPCs), and others (e.g., hearing disorders—50 PRCs, untreated visual deficits—12 PRCs, alcohol disorder—3 PRCs, caffeine effect—2 RPCs, etc.).

The diagnostic work-up includes a detailed information gathering as mentioned above, a specific phenomenological syndrome definition by a cognitive-behavioral-functional team of experts, detecting as many as possible etiological components by neurologists, geriatricians, psychiatrists and consultants, preparing integrated differential diagnosis of every cognitive, behavioral and functional syndrome and sub-syndrome, preparing recommendations for auxiliary tests, preparing best treatment available recommendations and continuing exploratory and dynamic follow up and case management.

According to an embodiment of the invention, some important implications of the INETAP method include the potential for stabilization and improvement of SLOD, offering a practical and optimistic work-up process, more adequate treatment principles which are derived from the complex system features of SLOD, a possibility for effective pre-symptomatic and para-symptomatic prevention programs, lowering costs, improving the current research questions, hypotheses and methodology.

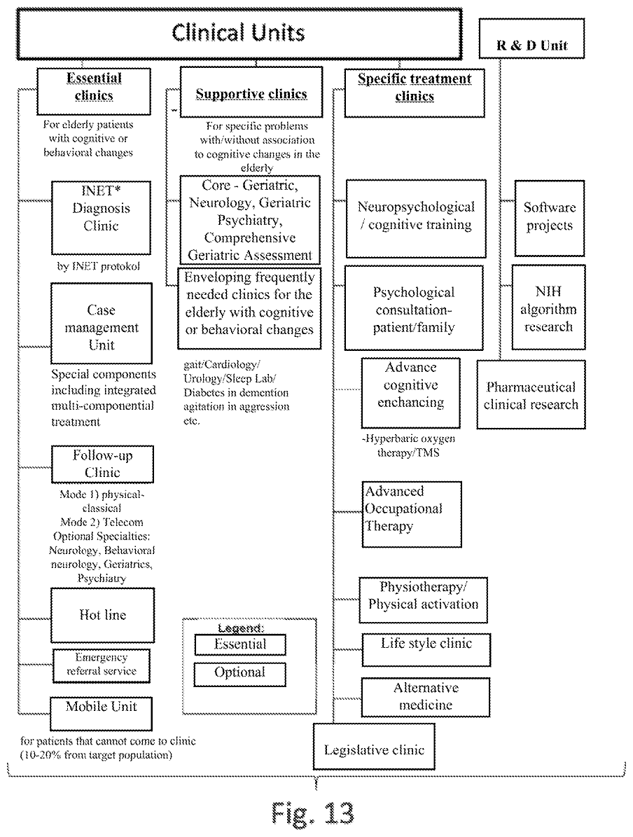

According to an embodiment of the invention, the INETAP method may involve the following procedures (as shown with respect to FIG. 11):

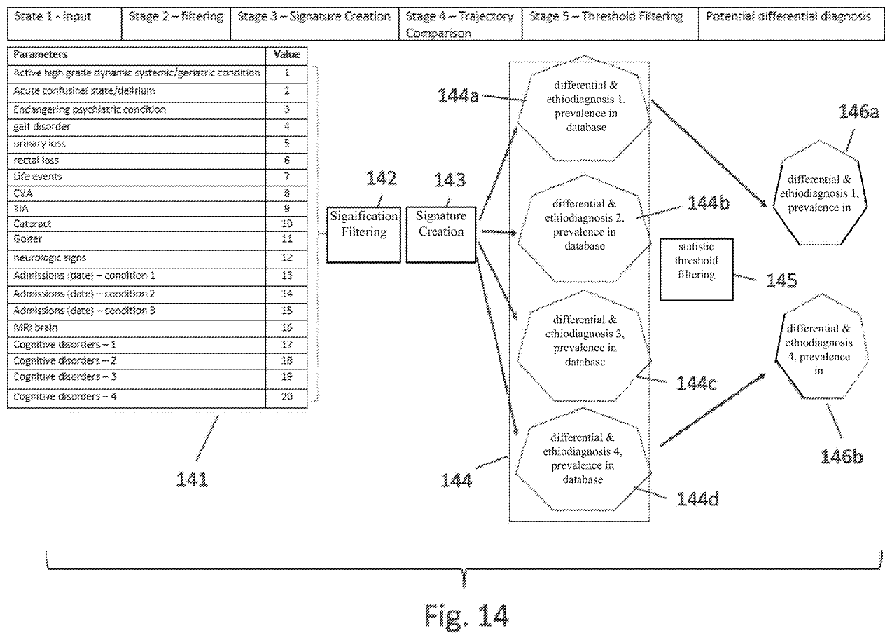

At first (step 101), receiving, as an input, data relative to medical history and examinations of each specific patient 1014 (e.g., paramedical interviews 1011, neuropsychological tests 1012, and medical checks and tests of a patient 1013). The received data may comprise genetics, age, resilience, lifestyle, and homeostasis & allostasis processes in accordance with multimorbidity-vascular disorders, multimorbidity-systemic disorders, and multimorbidity-geriatric disorders. Next (step 102), processing said received data by applying INETAP method related algorithms and verifying whether said processed data is sufficient (block 1022) for identifying advanced Dementia PRC or early-stage dementia PRC. If the data is not sufficient (block 1023) returning to the input data step for obtaining additional data. The INETAP method related algorithms will be described in further details hereinafter with respect to FIG. 12. Next (step 103), outputting data indicating whether advanced dementia PRCs identified 1031, pre-dementia (i.e., early-stage) identified 1034, no dementia identified 1033, or Dementia without treatment horizon identified 1032. Next (step 104), for advanced dementia PRC and pre-dementia providing treatment recommendations (TRs).

The algorithm provides an assessment of dementia and pre-dementia states (e.g., aiding in the diagnosis of Mild Cognitive Impairment and Subjective Cognitive Decline), through an algorithm that utilizes detailed interrogation procedure, identifying typically unknown categorical multimorbid and associated brain perturbative conditions, with the specific potential contribution to decline, diagnosis of all co-existing phenomenological syndromes and sub-syndromes of presenting dementia, previous cases, and new external research that proves the contribution of new factors to decline.

The algorithm enables estimation of Potentially Remediable Conditions (PRC) and specific Brain Perturbative Conditions (BPCs), that affect dementia or the pre-dementia state of a patient (such as hypertension, borderline heart failure, etc.).

Therefore, the method proposed by the algorithm is based on PRC. It is important to emphasize that the objective of looking for hidden condition is designed to create a chain of information-gathering, starting from the complaint and carving the way to specific conditions.

FIG. 12 schematically illustrates a system 120 for identifying treatable and remediable factors of Dementia and aging cognitive changes, according to an embodiment of the invention. System 120 comprises a Decision Engine (DE) 206, a Decision Analysis Engine (DAE) 208, a Decision Algorithm (DA) 207, a database (DB) 204, a User Interface (UI) 202, and a Data Gathering Verification (DGV) module 205.

The role of DE 206 is to transform collected data into preferred co-existing diagnoses and PRCs. In order to do so, DE 206 collects the data from a plurality of data sources such as INETAP 203 (i.e., data received from one or more medical sources, such as clinics database, questionnaires, clinical evaluation, etc.), from IoT and from Decision Analysis Engine (DAE). It then processes the information by the use of the DA 207, which analyzes information provided by DE 206.

The role of DAE 208 is to generate and update the DA 207. In order to generate or update DA 207, DAE 208 collects data from various sources, including the DB 204, new medical research 209, and medical comments made by clinicians. By the use of the collected data, DAE 208 creates the DA 207, to be implemented in DE 206.

Decision Algorithm (DA) 207 is the algorithm that generates Treatment Recommendations (TRs) and/or only PRCs based on incoming information received via the UI 202. It is able to create a comparison of Trajectories of medical and cognitive conditions (T) of patients 201 and compare them with previous cases in the database. Upon presenting the preferred PRCs 210 to clinicians for final decision and authorization, the statistical foundations of the trajectories are provided (e.g., displayed), so that clinician can make a knowledgeable decision. DA 207 is generated by DAE 208 and used until new updates are implemented.

DB 204 stores data of the various cases. In addition to medical history and selected preferred PRCs, it also includes updated data regarding the medical and cognitive state of patients 201 after evaluation and treatment have been performed.

According to an embodiment of the invention, in order to maintain privacy, the DB 204 can be divided into two main sections: the administration section that includes only the medical data, and the operator section that includes both medical and private data.

UI 202 can be used to display the main findings to the operator. For example, such findings can include proposed preferred PRCs 210, the statistic foundations thereof, and the medical foundations. The operator can accept or reject preferred PRCs according to his knowledge or other considerations.

DGV module 205 verifies that all needed medical and other information has been provided. According to an embodiment of the invention, this is obtained by (1) verifying that subjects for further investigation, as defined by the clinician, has indeed been provided and or (2) activating a procedure that compares the list of additional information, as defined by the clinician, with previous cases of high similarity—then prompting the information accordingly. For example—if the clinician suspects vitamin deficiency but he has not requested such a test, then the DGV module 205 can prompt the clinician to add the request for such test.

According to an embodiment of the invention, DA 207 is updated periodically with data from the DB 204, consisting of the performance of the cases, previously loaded to DB 204, analyzed with analytic tools, also used in other Big Data systems. For example, such analysis can be conducted in various ways:
a. Artificial Intelligence (AI) Building utilizes various models of analysis T and comparing outcomes. For each model, Hyper Parameters will be taken into consideration. Performance can be validated by K-fold Cross-Validation. Models to be considered during the process: (1)—“instance-based k-nearest neighbors (KNN) algorithm” or other suitable non-parametric supervised machine learning algorithms, which allows the generation of recommendations based on historical cases—based on the closest cases possible within K potential patients, (2)—“Random Forest”—quantitative or qualitative, (3) “XG Boost”—quantitative or qualitative, eta, Gamma, (4) Deep Learning—based on networks based on various architectures
b. Relate to the update as a Supervised Multi-Label classification. In this way, a model that resembles decisions previously made based on a specific tool-box of treatments is generated. For example, by the use of software languages “R” and “Python”, engaging various libraries such as—Caret, data.table, ggplot2, mlr, Fselection, RKeras, (for “R”) and for Python—pandas, NumPy, sklearn, Keras, TensorFlow 2.0. The process generates an overview of the required solution, origins of information, processing, and outcomes. Data for overlapping elements are cleaned, and a report is generated that allows medical staff to operate accordingly and conclude which final preferred PRCs are authorized for use.
c. New medical research: such new research may result in a change of preferred PRCs towards a less prevalent path, though with better medical foundations.

According to an embodiment of the invention, upon enrollment, the INETAP method uses the algorithms to define risk profile, and proximity is analyzed for each incoming dataset post evaluation requiring a change or updating of the treatment plan. The algorithm uses the correlation between medical, geriatric, neurological, cognitive, psychiatric, and psycho-social detailed historical events—and the trajectory of the current decline, to define the full spectrum of presenting cognitive syndromes.

The various methods of updating create a situation of overriding the previous algorithm in the sense that it changes the trajectories may be of lower statistic value, though with additional information, justifying the change in the course of action.

Issuance of treatment recommendation—with this additional method of issuing preferred PRCs, the following process is engaged:
a. INETAP PRCs are provided to DE 206;
b. DE 206 creates a pattern, based on the information provided; and
c. Then DE 206 compiles TRs, based on the response from DA 207.

According to an embodiment of the invention, by using the method proposed by the INETAP's algorithm, the medical personnel can easily provide medical assistance to a patient based on the PRC. In addition, the method proposed by the algorithm not only provides PRC, but also provides a trend indication for severity dynamics whether an individual is improved, deteriorated, or even approaching a dementia state.

The underpinnings of the development of the algorithm relative to its novelty, features, and technical contributions are as follows:

The development of the algorithm relates to a method, system and medium for modeling and controlling processes to interrogate data for the PRCs (Potentially Remedial Conditions), BPCs (Brain Perturbative Conditions), DHMOs (Diseases Hierarchical Multilevel Ontologies), treatment protocols and additional testing required to provide a cogent diagnosis of the state of cognitive decline. This method uncovers the interrelationships of PRCs, pattern recognitions and disease hierarchical determinations to cognitive decline and correlates the exacerbation of PRCs to cognitive decline. This novel correlative technology provides a detailed analysis of the interrelationships of etiological, neuropsychological parenchymal cellular, and subcellular analysis relative to determining the patterns of cognitive decline.

More specifically, the algorithm relates to modeling techniques that are adaptive to analysis of the empirical data points collected during/after the cognitive decline screening and diagnosis process implementation.

As in the implementation of conventional dementia screening and predictive models, use a lookup table, without using a mathematical model, is used to determine the best combination of input parameters to control the characteristics of dementia screening. This technique, however, often requires collecting and storing an enormous corpus of experimental data obtained from numerous real-time trials. These drawbacks make this example technique a complicated, inaccurate, time-consuming, and costly procedure.

According to some embodiments of the invention, the method advantageously overcomes the above-described shortcomings of the aforementioned techniques. More specifically, some embodiments provide a system, method, and medium for adaptive control models that use empirical data points.

In general, according to some embodiments of the invention, the algorithm first defines an input domain, which encompasses substantially all (if not all) possible values of input parameters. The input domain can then be divided into smaller regions called cells. In each cell, extreme values are identified (e.g., nodes, representing four corners of a two-dimensional input domain). A mathematical equation, called an objective function, is minimized based on the cells and extreme values of predicted and empirical output characteristics. By minimizing the objective function, a predictive model is obtained. By minimizing a different objective function related to the output characteristics of the predictive model, a set of values for input parameters can be obtained given the desired output characteristics.

In particular, a method according to one or more embodiments of the algorithm includes the steps of identifying one or more input parameters that cause a change in an output characteristic of a process, defining global nodes using estimated maximum and minimum values of the input parameters.

In order to differentiate critical empirical data from less critical ones, the coefficient Wi in the objective function can be adjusted based on, for example, heuristic information/knowledge. This makes the objective function respond, as precisely as possible, to the latest empirical data point, while being less responsive toward the earlier empirical data points.

The equilibrium positions reached by the virtual systems described above represent the minimization solution of the objective functions. In other words, the task of finding the minimum of the objective function is reduced to the task of determining the dynamic process of identification of PRCs. Analogizing the minimization problem into the language of “mechanics”, namely decision trees relative to the task of merging screening data into actionable PRCs and the relevance to cognitive decline.

Moreover, using the algorithm, new data (empirical data) points are obtained in the course of the process. Therefore, the system can be constantly updated according to the newly obtained data points. It follows that embodiments of the algorithm are adaptive to empirical data.

According to an embodiment of the invention, the system may serve as a guidance system:

This consists of various stages of accompanying clinicians through an interrogation process to find new information of relevance. For example, the process may entail several stages, as follows, for a guided journey towards finding remediable conditions:

TABLE 1

| No. | Stage | Activity | Participant | System's Server |
|---|---|---|---|---|
| 1 | Initial | View a landing | Patient | Establishes |
| | contact | page, fill in | | patient file |
| | | contact information, | | (e.g., under |
| | | and click | | HIPAA/GDPR |
| | | “OK” to | | or other |
| | | receive further | | data protection |
| | | information | | regulations) |
| 2 | Preparations | Uploads medical | Patient | Stores uploaded |
| | | history five | | information and |
| | | years back, | | generates |
| | | current medical | | questionnaires |
| | | state, drugs taken, | | |
| | | hospitalizations, | | |
| | | imaging, etc., | | |
| | | introduction | | |
| | | questionnaire | | |
| 3 | Nurse | Vital signs, | Nurse/ | Stores uploaded |
| | Consultation | initial impression, | Patient | information and |
| | | information | | generates |
| | | validation, further | | questionnaires |
| | | explanations to the | | |
| | | patient/family | | |
| 4 | Neurologic | Neurologist runs | Neurologist/ | Generates |
| | evaluation | a series of | Patient/ | a list of |
| | (session 1) | neurologic | | proposed |
| | | tests and | | tests and |
| | | interrogations | | specialist |
| | | supported by | | consultations |
| | | the system | | for further |
| | | | | interrogation |
| 5 | further tests | Further checks | Patient/ | Generates a |
| | & checks | & tests are | Nurse | list of further |
| | | needed to complete | or secretary | checks and tests |
| | | the overall | | |
| | | picture, such as | | |
| | | MRI, Sleep Lab, | | |
| | | etc., and | | |
| | | specialists | | |
| | | consultation | | |
| 6 | Neuro- | Memory tests, | Neuro- | Stores uploaded |
| | psychologic | concentration, | psychologist | information |
| | evaluation | language/speech, | | |
| | | visual | | |
| | | perception, | | |
| | | managerial | | |
| | | functioning, | | |
| | | understanding, | | |
| | | and judgment | | |
| 7 | Data | Summarizing | Neurologist | Outputs: |
| | analysis | findings, Edit | | Intermediate |
| | | Intermediate | | summary |
| | | Report, including | | report (final in |
| | | background | | relatively |
| | | information, main | | simple cases) |
| | | possible causes, | | |
| | | conditions to | | |
| | | rule out, | | |
| | | contributing | | |
| | | factors, | | |
| | | and more. | | |
| 8 | Neurologic | Introducing | Neurologist/ | Generates |
| | evaluation | findings to the | Patient | differential |
| | session (2) | patient and | | diagnosis |
| | | discussing further | | of background |
| | | steps needed. | | diseases |
| | | | | and final |
| | | | | treatment plan. |
| | | | | Create a |
| | | | | list of action |
| | | | | items/tests |
| | | | | related to |
| | | | | further steps |
| | | | | Provide |
| | | | | foundations for |
| | | | | Intermediate |
| | | | | Report. |

In view of table 1 above, at the Preparation stage (No. 2), the system uses the algorithm to generate a set of questionnaires, including medical history, current medical state, cognitive decline tests, etc. At the Nurse Consultation stage (No. 3), the algorithm generates a set of nurse questionnaires, vital signs, summary of previously collected information, etc.

At the Neurologic evaluation stage (session 1) (No. 4), upon receiving neurologic consultation results and information intake from previous stages, the algorithm compares with previous cases and generates a list of proposed tests and further consultations. Optionally, a clinician may add tests based on his own experience.

At the further tests & checks stage (No. 5), based on previous cases of high similarity, the algorithm generates a list of medical and other tests needed to complete the full analysis.

At the Data analysis stage (No. 7), the algorithm generates an intermediate report. The report may reflect an analysis of inputs clustered as background information, main possible causes, conditions to rule out, and contributing factors. In simple cases, this may be the last step.

At the Neurologic evaluation stage (session 2) (No. 8), the clinician verifies the outcomes facing his medical background and training. A clinician can also add his own recommendations. The objectives of this stage are: (1) to ensure clinicians' consent since he is taking the responsibility, (2) to allow a level of deviation from recommendations, thereby encouraging new data to hit the system, and (3) to accommodate to personal limitations, e.g., the lack of ability to follow certain recommendation due to physical disabilities.

According to an embodiment of the invention, the system may comprise one or more of the following additional operational modes.
a. Simulation—prior to deciding which preferred PRCs to accept, the clinician can prompt the system 200 for an analysis of the course chosen. In such a case, the system will compile a simulation of how such a case may evoke. The simulation is based on the analysis of previous cases of defined similarity or statistic proximity. Such similarity can be modified in statistical terms by defining one of several parameters, such as Standard Deviation, age, state of decline, medical situation, and more.
b. Analyze previous cases with DE 206—by the use of updated algorithms, patients (i.e., subjects) that were previously diagnosed can be re-evaluated and approached. For example, the way this is conducted is by feeding the very same information to DE 206 and comparing new TRs that were generated with those previously issued. This is very different from the regular setup since patients do not need to approach the clinic for assessment. In case a significant change has been found, the clinician can approach the patient to offer updated preferred PRCs 210.
c. The use of the Internet of Things (IoT), wearable devices, or other external or new information—in case there is a requirement to analyze a certain thesis, this can be performed by monitoring patients and see if certain values change and to what extent. If such values were achieved, an alert could be activated to call upon action by the operators. The external information may be changed in certain values or new indications. New indications could be but are not limited to new medical events, changes in cognitive state, and/or new psychological events. One example is if there is a concern that a person has sleep apnea or may develop such a condition in the future. In such a case, a monitoring device may be connected to the patient. In case the condition indeed is localized, an alert is communicated to the clinician, who can take further action.
d. Biomarkers (BM)— use of biomarkers can be applied to create an additional indication of the cognitive state. This improves performance since the improvement is reported without a need to conduct cognitive analysis and testing of the patient. Hence, the report could be more accurate and be applied at earlier stages, where the patient is not yet aware of the potential change. The way it works: The value of BM is added to the various medical values. In order to monitor the change, BM can be used as an early indication, which requires by far less skilled manpower. Once the new value is provided to the system, a calculation is performed, and a decision for further action can be taken.
e. Drug and new treatments' development—another application of the solution is in support of drug development- and implementation. The need for this is due to the complexity of medical situations prevalent within the elderly, as previously described.
f. Simulate drug development—assuming the drug is expected to generate a certain change in the medical values. Such change can be defined as a new case and simulated accordingly. For example, the way to perform this simulation: prior to deciding which preferred PRCs to accept, the clinician can prompt the system 200 for an analysis of the course chosen. In such a case, the system will compile a simulation of how such a case may evoke. For example, the simulation is based on the analysis of previous cases of defined similarity or statistical proximity. Such similarity can be modified in statistical terms by modifying one or several parameters, such as Standard Deviation, age, state of decline, medical situation, and more—to create a simulation that reflects the case in a better way.
g. Define a suitable drug composition—whether for personalized drugs or the use of one or more drugs—prior to deciding which drugs to subscribe, the clinician can simulate the implications of such a drug. This is conducted by modifying expected medical and other values, which are expected to change, in lieu of the considered drug composition. The simulation will then display the change of course, based on statistic proximity of cases, thereby allowing optimizing the drug composition.
h. Pre-symptomatic—Para Clinical Warning—another application of the system is in searching for pre-warning data: One of the biggest challenges is to find indications at preclinical stages. However, at that stage, the absence of complaint of cognitive or behavioral nature makes it difficult to create a starting point for examination.
   The objective—to localize enough similarity to create a kick-in situation, where a patient is indeed diagnosed as being of high risk of developing a cognitive decline in the foreseeable future. By the use of Pattern Recognition, PRCS can be compared to PRCs found at Post Clinical stages, and the level of a match can be displayed.
   The Process—In order to cope with this situation, the following action is proposed: Facing the increase of Multi Morbidity (MUM) with the increase of age, the proposed method of analysis uses MUM found in other cases in the DB that are categorized as Post Clinical, with Mild Cognitive Impairment. The PRCs found at this stage are mapped and used as target PRCs for earlier stages. Each PRC is also given a risk rate, which reflects its contribution to further decline. Those values can be found by analyzing which PRCs were indeed treated and what the impact was. Cases at the preclinical stage can now be analyzed: New cases are examined by INETAP in order to locate PRCs. Once PRCs have been located, a PRC Signature (PRCSG) is created. It should be noted that PRCSG contains less information than PRCs from post clinical stages.

According to an embodiment of the invention, one way to realize the comparison is by the use of 1:N, similar to fingerprint recognition, where the sample is compared to the entire database or parts thereof. Matches of high similarity are then displayed. This method allows the system to recognize such patterns of PRCs and prompts the clinician to address those PRCs, despite that the patient is not aware of any cognitive decline.

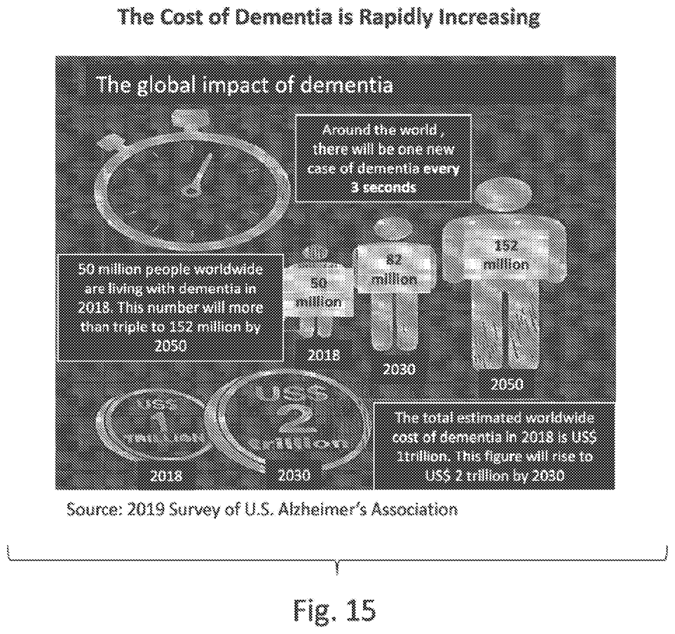

FIG. 13 schematically illustrates, in a block diagram form, clinical units, according to an embodiment of the invention. This figure shows the integration of a diagnostic clinic based on the INETAP method of the present invention. In this example, the clinical units may comprise essential clinics, supportive clinics, and specific treatment clinics. Feedback data (i.e., exploratory follow-up) from patients being treated according to the recommendation provided by the system of the present invention can be used to update the INETAP algorithm, thereby enabling the system to improve itself. Such improvement can be achieved by involving suitable machine learning approaches to provide a self-learning system.

All the above will be better understood through the following illustrative and non-limitative examples.

### Example 1—the Significance of Micro-Cognitive Phenomenological Findings on the Diagnosis and Treatment of SLOD

A right-handed 77-year-old person, a holocaust survivor, and retired high-rank municipality officer, was referred to an INETAP clinic (i.e., a clinic that uses the INETAP method) for a second opinion because of cognitive decline that was diagnosed as progressive degenerative Dementia most probably mixed type (AD and VD).

Data related to his medical history included hypertension, ischemic heart disease with a history of anginal pain, and congestive heart failure. Data related to his treatment included furosemide, metoprolol, and captopril. There were no other systemic or neurological deficits on the system review.

Physical examination was noncontributory except for supine and standing BP of 95/50 and 90/50, respectively. Heart rate (HR) was 82 bpm, regular. Motor-sensory neurological examination was negative.

His INETAP evaluation consisted of 2 stages.

### Stage 1: Assessment of Apparent Cognitive Syndrome—

The patient and his wife reported that he has less ability to concentrate and could not explain his thoughts or remember earlier conversations. The deficit has been progressive over the last two years. He was independent in daily activities except for those that needed verbal communication. Cognitive and behavioral symptoms reviews were negative besides the mentioned complaints.

Behavioral neurological and neuropsychological examination revealed a fully cooperative individual who showed appropriate affect and psychomotor activity. The speech was fluent. However, his responses to open questions were incomprehensible. The sequence of words made no sense, and the messages could not be understood. Some words sounded like neologisms, not existing in Hebrew, the language in which he was examined. He had good verbal comprehension, as was indicated by pointing and yes/no tasks. He also had full repetition ability. He could name visual objects flawlessly. His memory was thought to be preserved, as was indicated by episodic verbal and visual memory tests. Additionally, he was able to remember to perform long term orders, such as returning to examination a few days later to be examined by a specific person in a specific place. He was also able to remember shopping lists. Other cognitive domains, including insight, were intact.

### Comment to Stage 1:

SP has a progressive atypical transcortical motor apathetic syndrome with difficulty in spontaneous language production but preserved comprehension, repetition, and object naming. The apathetic syndrome could be localized to the left inferior posterior frontal area (peri-Broca). It did not seem to be degenerative non-fluent/agrammatic Primary Progressive Aphasia (nfaPPA) in view of the fully preserved object naming ability, absence of apraxia of speech, and lack of any understood cluster of words. Additionally, there was no effort of speech, no pauses, the rate of the production was quite high, and the length of clusters was normal. Few reported events of speech arrest were also atypical of nfaPPA. In addition, there was no evidence of Dementia.

A full diagnostic work-up was initiated in view of the severe hypotension and the need to exclude active PRCs.

### Stage 2: Establishing Differential and Etiological Diagnosis

A specific syndromal work-up was performed because the impression was that the symptomatology could have resulted from a specific PRC.

The preserved object naming ability in the face of the garbled and incomprehensible sentences defined the disorder as a sentence production deficit. To further analyze this phenomenology, the cognitive five levels model of sentence production deficit was reviewed (Garrett, 1985). The Functional Level Representation is the first post-message level and has a role in logical and syntactic processing. It includes nouns and verbs, lexical selection, and functional argument structuring. Since, in this case, nouns were selected without difficulty, we added the evaluation of verb naming. The patient could not name verbs (only 2% correct). This was in contrast to the preserved naming of nouns (98% correct).

Additionally, there were a few grammatical errors. Thus the cause of the sentence production deficit was specifically related to the verb anomia.

Etiological work-up, including ultrasound of the carotid arteries, was negative except for low blood pressure on ambulatory 24-hour monitoring. This was an authentic change of 2 years—the symptomatic period—since reviewing values from until this time showed values of about 140-150/75-85 mmHg. The decreased values were revised to begin after the addition of afterload reduction treatment by captopril immediately after cardiac cauterization. Also, his brain CAT showed heavily calcified left dorsolateral frontal branches of the middle cerebral artery (MCA). Brain FDG-PET showed a localized hypometabolic area in the left posterior middle and inferior frontal gyri, which are included in the frontal dorsolateral border zone. The diagnosis thus was chronic progressive verb anomia due to hypoperfusion ischemia.

In coordination with the cardiologist, the dosage of the captopril was lowered, and blood pressure values arrived around 135/70. The speech improved significantly and stayed stable for three years.

General comment—micro-phenomenological analysis identified an isolated language syndrome that helped in excluding a degenerative brain disease. It also encouraged etiological work-up and specific regenerative treatment.

### Example 2—MEPC in the Evaluation of SLOD

A 78 years old very handyman, who was a security officer in a big cigarette factory, was referred to INETAP method evaluation because of a two years slowly progressive memory and functional changes that were diagnosed as Alzheimer's disease (AD). He and his wife complained about memory difficulties-forgetting meetings, losing significant articles at home, events of misidentification of familiar roads while driving, being less initiative, more apathetic, and a little impulsive. It was more difficult for him to manage his finances. However, he continued to be fully independent, though neglecting home arrangements. He complained of fatigue and excessive daytime sleep.

He was examined six months before and had MMSE 26/30 and MoCA-22/30. He was diagnosed with Alzheimer's disease (AD) and treated with Donepezil and Memantine for six months without improvement.

System review-disclosed mild hearing loss, sleep difficulties (snoring, difficulty in sleep maintenance), fatigue and excessive daytime sleepiness,

He Has a background of essential hypertension, dyslipidemia, lower urinary tract symptoms (LUTS), and past history of vitamin B12 deficiency ten years ago.

A positive finding on evaluation included decreased attention, decreased episodic delayed recall verbal with preserved recognition memory, preserved spatial memory, decreased complex visual memory, decreased working memory, calculation ability, decreased phonemic and semantic word generation, abstraction, and set-shifting ability. He was perseverative in the Multiple Loop task. He was a little impulsive. He has preserved naming ability, language functions, semantic knowledge, map knowledge, face, and objects spatial recognition distribution of attention. MMSE was 28/30, CDR-0.5/3, and GDS-1/15. Motor sensory neurological, as well as systemic examinations, were noncontributory except for morbid obesity and ischemic oculopathy.

The patient was asked to perform a comprehensive vascular, blood tests, CAT scan, hearing test, EEG, and urinalysis. The tests showed—Uncontrolled systolic and diastolic hypertension (24 h ambulatory monitoring—awake period systolic—mean-182+/−27, max-230, min-146, diastolic-94, 29.6, 168, 57 respectively; asleep period-systolic-mean-149+/−15.9, max182, min-131, diastolic-81+/−14.3, 122, 61 respectively). High LDL, Vitamin D insufficiency, Low normal range vitamin B12. A brain CAT scan showed marked periventricular leukoaraiosis. Polysomnography revealed a severe obstructive SAS (AHI-36/h) and nocturnal hypoxemia (O2Sat>90%-11% of the sleeping time).

The patient was diagnosed as suffering from MCI. The main cause was subcortical ischemic vascular, due to uncontrolled hypertension and hyperlipidemia. Major contributing factors were mainly SAS, and nocturnal hypoxemia. Additional contributing factors findings include low B12, vitamin D, and decreased hearing.

He was recommended to treat these disorders. As a result, the sleep disorders improved significantly, and the cognitive deficit was stabilized for two years.

Comment—This patient was first diagnosed with AD due to the progressive memory decline that was perceived as the dominant complaint and the age contingency. As a result, no treatment for PRCs was offered. The Donepezil did not change the progressive course. In fact, this approach did not leave any hope for the future with a progressive deterioration quite sure, for example, due to uncontrolled hypertension and sleep disorders.

The INETAP method was directed to the multi-etiological phenomenological complexity (MEPC) features of SLOD. Due to the assumed MUM existence, there was high sensitivity to the phenomenological symptoms and findings. Thus, the dysexecutive components with the preservation of frequent AD features like semantic knowledge and naming as well as the presence of fatigue—suggested several phenomenological sub-syndromes. This encouraged a thorough diagnostic work-up that disclosed seven PRCs, four of them quite major. The complexity basis of the SLOD in this patient stimulated the all-PRCs effective treatment.

All the above description and examples have been given for the purpose of illustration and are not intended to limit the invention in any way. Many different methods, electronic and logical elements can be employed, all without exceeding the scope of the invention.

1. A computer-implemented method for identifying treatable and remediable factors of Dementia and aging cognitive changes, comprising:
a) Receiving, using a processor, data relative to medical history and examinations of a subject;
b) Processing, using the processor and one or more machine learning algorithms, said received data to identify patterns of advanced dementia Potential Remediable Condition (PRC), and other data related to the subject's medical condition; and
c) Outputting data for aiding in the diagnosis of one of the following: dementia PRCs, pre-dementia PRCs, no dementia/pre-dementia, or Dementia without treatment horizon.

2. The method according to claim 1, further comprising outputting recommendations in accordance with symptoms of pre-dementia or Dementia PRCs.

3. The method according to claim 1, further comprising outputting recommendations in accordance with existing risk factors for no dementia/pre-dementia or Dementia without treatment horizon.

4. The method according to claim 1, wherein the data relative to medical history comprises detailed cognitive, behavioral, functional, neurological, psychiatric, lifestyles, psychosocial, medical, and geriatric information.

5. The method according to claim 1, wherein the data relative to examination comprises behavioral neurology, neuropsychology, psychogeriatric, neurology, psychosocial, medical, and geriatric information.

6. The method according to claim 1, wherein applying the algorithm comprises: a) processing data received from different levels of pathogenetic causality of Late-Onset Dementia (LOD) Syndrome Complex and distal brain molecular and cellular processes; b) identifying pathological changes in accordance with said processed data; and c) providing symptomatic LOD.

7. The method according to claim 1, further comprising providing statistical foundations of preferred PRCs decisions, thereby making it easier for clinicians to rely on preferred PRCs, allowing a faster authorization of issued preferred PRCs, shortening the training duration of a medical team, and considering and integrating new published worldwide relevant research.

8. A system for diagnosing and preventing dementia syndrome, comprising:
a) at least one processor; and
b) a memory comprising computer-readable instructions which, when executed by the at least one processor, causes the processor to execute a Potential Remediable Condition (PRC) agent, wherein the PRC agent:
i. receives data relative to medical history and examinations of a subject;
ii. processes said received data by applying machine learning algorithm to identify patterns relative to advanced Dementia PRC; and
iii. outputs data for aiding in the diagnosis of one of the following: Dementia PRCs, pre-dementia PRCs, no dementia/pre-dementia, or Dementia without treatment horizon.

9. The system according to claim 8, wherein the PRC agent enables:
to create the foundations of creating more sophisticated thresholds for further decisions and actions;
to create repeatability of decisions of preferred PRCs; and
to provide the statistical foundations of preferred PRCs decisions,
thereby making it easier for clinicians to rely on preferred PRCs, allowing a faster authorization of issued preferred PRCs, shortening the training duration of the medical team, and considering and integrating new published worldwide relevant research, and allowing an external information feed.

10. The system according to claim 9, wherein the external information feed is received from wearables and the Internet of Things (IoT).
